(19) United States
(12) Patent Application Publication
(10) Pub. No.: US 2018/0204189 A1
(43) Pub. Date: Jul. 19, 2018

(54) Method for Providing Product Information and Support Services

(71) Applicants: Manuel A Espinoza, Irvine, CA (US); Tsong Shyan Wang, New Taipei City (TW)

(72) Inventors: Manuel A Espinoza, Irvine, CA (US); Tsong Shyan Wang, New Taipei City (TW)

(21) Appl. No.: 15/922,630

(22) Filed: Mar. 15, 2018

Related U.S. Application Data
(63) Continuation-in-part of application No. 14/856,762, filed on Sep. 17, 2015, now abandoned.

(51) Int. Cl. G06Q 10/00; G16H 40/60; G06F 8/65

(52) U.S. Cl. CPC G06Q 10/20; G06F 8/65; G16H 40/60

(57) ABSTRACT

A method for providing product information and support services is performed by: providing a medical imaging equipment with a QR code and identification data, manufacturer accounts, a user account, a secure server for managing the manufacturer accounts and the user account and a portable computing device with a QR code scanner; communicably linking the user account to the secure server through the portable computing device; receiving the identification data by scanning the QR code with the QR code scanner; comparing the identification data to the manufacturer accounts in order to identify a matching manufacturer account associated with the medical imaging equipment; communicably linking the user account and the matching manufacturer account through the secure server; generating a viewable profile for the medical imaging equipment on the secure server; and enabling the user account or the matching manufacturer account to update the viewable profile with a notification or a log.

The current application claims a priority to the U.S. patent application Ser. No. 14/856,762 filed on Sep. 17, 2015.

### FIELD OF THE INVENTION

The present invention relates generally to a product maintenance method. In particular, the present invention introduces a method for providing product related information and providing support services.

### BACKGROUND OF THE INVENTION

With time, the number of items owned by an individual has increased tremendously. The convenience factor and the developments in technology are among the main reasons for the increased number of purchases. The large number of purchases has resulted in the individual paying less attention to maintaining a single item. The lack of attention can be especially disadvantageous if a particular item has a defect or other related issue. The present invention introduces a method to notify the user regarding each of the products in a timely manner. As a result, the user is guaranteed to get maximum use of a purchase.

Most companies introduce updates to their devices in a periodic manner. However, due to time constraints and similar reasons the user may not be able to keep up with all of these updates. The user may prefer to have some updates and also prefer not to have certain updates. Moreover, since most of these updates are not sorted in a particular manner, the probability of the user failing to see a preferred update is comparatively high. Therefore, the need for a method of organizing the available updates is clearly evident.

As seen on many marketing websites, the user needs to create an account to get updates on a particular product or receive support for the particular product. The process of creating an account can be both time consuming and stressful to the user. The present invention addresses the issue by providing the user with a common login which can be utilized to access each of the required marketing websites.

Certain devices require maintenance regularly. As an example, a machine that requires constant maintenance. Since the machine is usually positioned at a particular location, maintenance personnel may have difficulty in locating the particular location. The present invention addresses the issue by providing exact coordinate locations for the machine. As a result, the maintenance personnel can conveniently access and repair the machine.

As experienced by many individuals, the need to remember service dates and other maintenance related information can be difficult. For instance, the user may have difficulty in remembering the date for the next oil change for an automobile. The present invention allows the user to maintain a product related log which notifies the user in a timely manner. In this instance, the present invention notifies the user in a timely manner regarding the oil change. The product related log is also useful to portray the history of the product. As an example, if the user is willing to sell the product on a future date, the product related log can be used to provide product information to the potential buyer.

The objective of the present invention is to address the aforementioned issues. In particular, the present invention intends to constantly notify the user of updates, recalls, and other maintenance information related to personal property. In order to notify the user, the present invention establishes a line of communication between the seller and the user. The line of communication is then utilized to notify the user regarding the updates, recalls, and other related maintenance information.

### BRIEF DESCRIPTION OF THE DRAWINGS

FIG. 1 is a flowchart illustrating a basic overall process of a method for providing product information and support services of the present invention.

FIG. 2 is a flowchart illustrating receiving a visual image and location information of the method of the present invention.

FIG. 3 is a flowchart illustrating posting a repair completion log on a viewable profile of the method of the present invention.

FIG. 4 is a flowchart illustrating posting recall information for a medical imaging equipment of the method of the present invention.

FIG. 5 is a flowchart illustrating posting update notifications on the viewable profile of the method of the present invention.

FIG. 6 is a flowchart illustrating installing software upgrades of the method of the present invention.

FIG. 7 is a flowchart illustrating installing warranty notifications of the method of the present invention.

FIG. 8 is a flowchart illustrating posting a checkup notification of the method of the present invention.

FIG. 9 is a flowchart illustrating requesting technical information from a matching manufacturer account of the method of the present invention.

FIG. 10 is a flowchart illustrating posting a product review for the medical imaging equipment of the method of the present invention.

FIG. 11 is a flowchart illustrating utilizing a trouble ticket of the method of the present invention.

FIG. 12 is a flowchart illustrating rendering the technical information comprising a repair manual of the method of the present invention.

FIG. 13 is a flowchart illustrating rendering the technical information comprising a plurality of highly-rated knowledge base articles of the method of the present invention.

FIG. 14 is a flowchart illustrating rendering the technical information listing locations of other installed devices similar to the medical imaging equipment of the method of the present invention.

FIG. 15 is a flowchart illustrating providing a magnetic resonance imaging (MRI) equipment as the medical imaging equipment of the method of the present invention.

### DETAIL DESCRIPTIONS OF THE INVENTION

All illustrations of the drawings are for the purpose of describing selected versions of the present invention and are not intended to limit the scope of the present invention.

The present invention introduces a method to link a manufacturer with a customer in order to provide product related support and services. By utilizing the present invention, the manufacturer can maintain a line of communication with each of the customers. On the other hand, by utilizing the present invention the user is aware of all updates made available by the manufacturer.

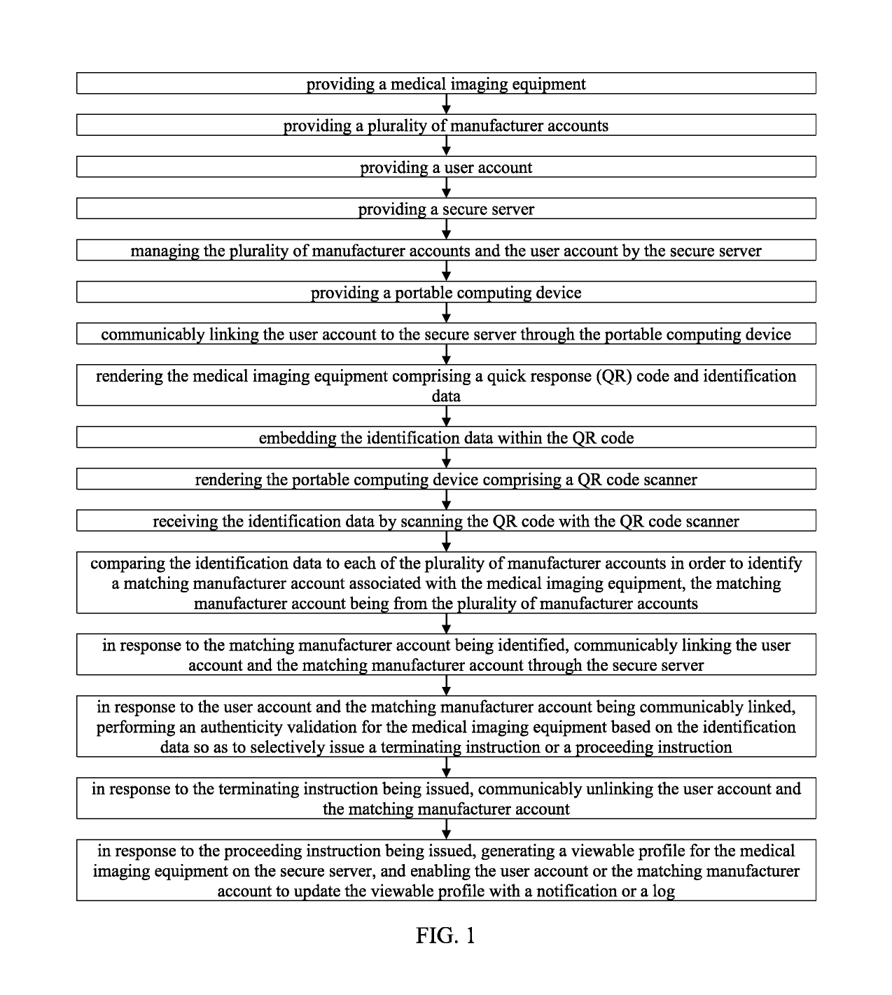

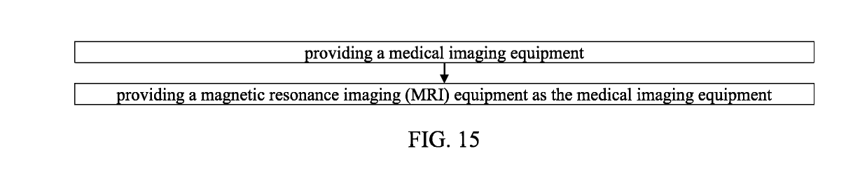

As described above, the present invention provides a platform that creates a two-way communication between the manufacturer of a product and the consumer of the product. In order to do so, the present invention provides a medical imaging equipment, a portable computing device, a secure server, a plurality of manufacturer accounts and a user account as seen in FIG. 1. The secure server is used to manage the plurality of manufacturer accounts and the user account. Each of the plurality of manufacturer accounts contains manufacturer information which can be, but is not limited to, contact information, location information, and other comparable information. On the other hand, the user account contains user information regarding the user of the medical imaging equipment. More specifically, user information which can be, but is not limited to, gender, age, contact information, and any other demographic information is included in the user account. Information from the user account is utilized by the plurality of manufacturer accounts to provide the most relevant information to the user account. As an example, if the user is attending college, the plurality of manufacturer accounts can provide information and deals that can relate to college students. All information received from the user account and the plurality of manufacturer accounts are saved on the secure server. In the process of connecting to the plurality of manufacturer accounts, the user utilizes the portable computing device. More specifically, the user account is communicably linked to the secure server through the portable computing device such that the user account can access information stored on the secure server whenever the need arises. Specifically, the medical imaging equipment is a magnetic resonance imaging (MRI) equipment, as shown in FIG. 15. The medical imaging equipment comprises a quick response (QR) code and identification data. The portable computing device can be, but is not limited to, a mobile phone, a laptop or any other comparable device. The portable computing device comprises a quick response (QR) code scanner. The present invention initially receives the identification data for the medical imaging equipment by scanning the quick response (QR) code with the quick response (QR) code scanner of the portable computing device. In order to do so, the identification data for the medical imaging equipment is embedded within the QR code. When receiving the identification data is complete, the present invention compares the identification data to each of the plurality of manufacturer accounts in order to identify a matching manufacturer account associated with the medical imaging equipment. In order to do so, the matching manufacturer account is selected from the plurality of manufacturer accounts. Upon finding the matching manufacturer account, the present invention communicably links the user account to the matching manufacturer account on the secure server. Next, when the user account and the matching manufacturer account are communicably linked, an authenticity validation for the medical imaging equipment is performed based on the identification data. Accordingly, a terminating instruction or a proceeding instruction is selectively issued. For example, the manufacturer could perform the authenticity validation for the medical imaging equipment, and thus provides the terminating instruction or the proceeding instruction. When the terminating instruction is issued, the user account and the matching manufacturer account are communicably unlinked, such that the user will not be notified anymore. On the other hand, when the proceeding instruction is issued, the entire customer support flow will be continued in such a way that the present invention generates a viewable profile for the medical imaging equipment on the secure server. When the viewable profile is generated, the present invention enables the user account or the matching manufacturer account to update the viewable profile with a notification or a log. In particular, the user is allowed to centrally manage personal belongings. As an example, the user account can contact the matching manufacturer account with the notification. Similarly, the user account can update the viewable profile with any medical imaging equipment related information by using the log. Similar to the user account utilizing the notification to contact the matching manufacturer account, the notification to update the viewable profile can also be utilized by the matching manufacturer account to contact the user account. Therefore, a two-way communication line is established between the user account and the matching manufacturer account. The two-way communication line can be used by the manufacturer for marketing purposes and also used in product development.

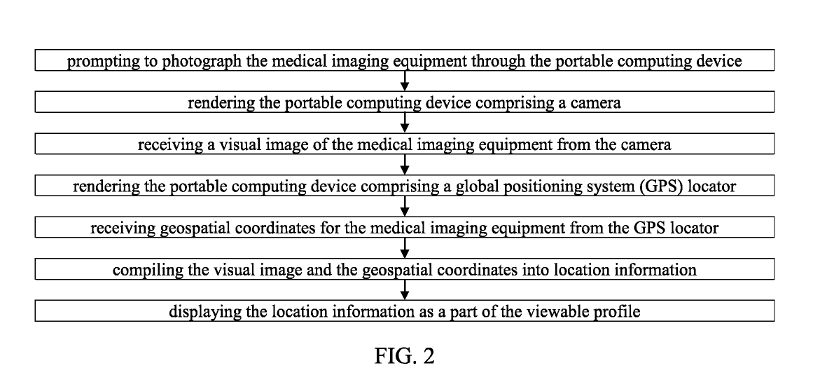

The present invention is intended to be used by the manufacturer to provide support and services to the customer. Different circumstances require different levels of support and service. As an example, certain issues of the medical imaging equipment may require expert knowledge making a technician visit mandatory. If the medical imaging equipment is located in a specific location, finding the specific location can be time consuming and stressful to the technician. As an example, if the medical imaging equipment is in a building complex, the technician may have difficulties in locating the medical imaging equipment conveniently. In order to address such issues, the present invention provides a global positioning system (GPS) locator and a camera for the portable computing device as shown in FIG. 2. The GPS locator is utilized to provide the exact location of the medical imaging equipment. On the other hand, the camera is used to provide visual information regarding the medical imaging equipment and the surroundings of the medical imaging equipment. In doing so, the present invention initially prompts the user account to photograph the medical imaging equipment through the portable computing device. Next, a visual image of the medical imaging equipment is received through the camera of the portable computing device. Simultaneously, the geospatial coordinates for the medical imaging equipment are received from the GPS locator. When received, the present invention compiles the visual image and the geospatial coordinates into location information for the technician. Resultantly, the technician can locate the medical imaging equipment conveniently since the location information is displayed as a part of the viewable profile. Referring back to the previous example, the user account uploads a photograph of the medical imaging equipment along with any other relevant photographs of the surroundings of the medical imaging equipment. The photograph of the medical imaging equipment and the relevant photographs are saved on the secure server through the viewable profile. Simultaneously, the geospatial coordinates for the medical imaging equipment are also saved on the secure server through the viewable profile. Therefore, the technician can conveniently identify the medical imaging equipment, regardless of the specificity of the location of the medical imaging equipment.

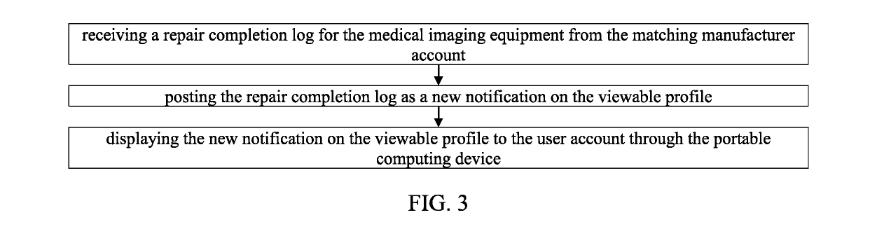

As discussed earlier, the present invention is used to constantly update information regarding the medical imaging equipment. For instance, if a repair is done on the medical imaging equipment by the matching manufacturer account, information regarding the repair is saved to the viewable profile for future reference. In order to do so, the present invention receives a repair completion log for the medical imaging equipment from the matching manufacturer account as shown in FIG. 3. The repair completion log can vary from one medical imaging equipment to another. When the repair completion log is received, the present invention posts the repair completion log as a new notification on the viewable profile. Next, the new notification is displayed on the viewable profile such that the user account can view the new notification through the portable computing device. Even though the repair completion log is received from the matching manufacturer account in the preferred embodiment of the present invention, the repair completion log can also be received from the user account in different embodiments of the present invention. In particular, if the user independently completes a repair for the medical imaging equipment, the user is allowed to post the repair completion log as the new notification to the viewable profile. Since the repair completion log is made available on the viewable profile, both the matching manufacturer account and the user account can access the repair completion log when information regarding the medical imaging equipment is needed. Additionally, the repair completion log can be used by the matching manufacturer account to understand a potential fault in the medical imaging equipment. For instance, if the matching manufacturer account identifies that the repair completion log addresses a single issue repeatedly, the matching manufacturer account can proceed to address the issue at a larger scale such as making recalls for the medical imaging equipment.

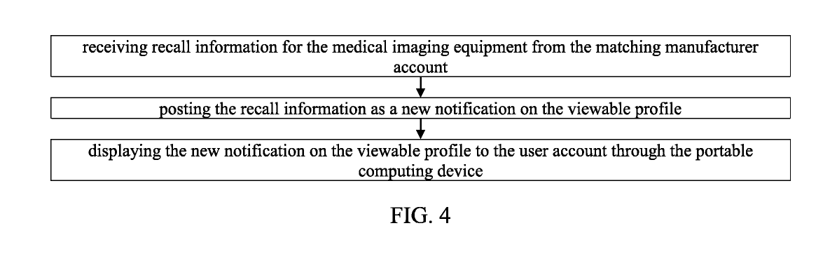

As illustrated in FIG. 4, the present invention can be used by the manufacturer to address an issue existing with the medical imaging equipment in a large scale. In the process of using the present invention to distribute recall information, the present invention initially receives recall information for the medical imaging equipment from the matching manufacturer account. When receiving is complete, the recall information is then posted on the viewable profile as a new notification. As a result, the new notification on the viewable profile is displayed to the user account through the portable computing device. Therefore, the present invention ensures that the user account is promptly notified regarding the recall information. According to the user's preference, the new notification regarding the recall information can be either accepted or ignored.

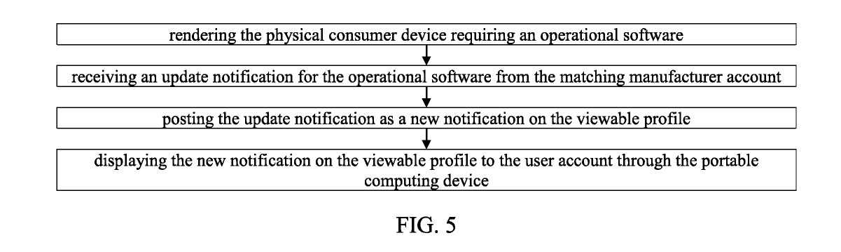

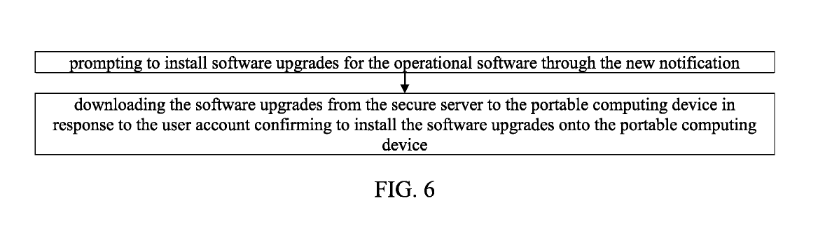

A majority of manufacturers provide updates for the medical imaging equipment in a periodical manner. As an example, the manufacturer of the medical imaging equipment provides software updates for the medical imaging equipment in a periodical manner. As shown in FIG. 5 and FIG. 6, if the medical imaging equipment requires an operational software, the present invention receives an update notification for the operational software from the matching manufacturer account. When the update notification is received, the present invention posts the update notification as a new notification on the viewable profile. When the new notification is displayed on the viewable profile, the user account can either confirm or decline the new notification by utilizing the portable computing device. More specifically, the present invention prompts the user account to install software upgrades for the operational software through the new notification. If the user account conforms, the software upgrades are downloaded from the secure server to the portable computing device.

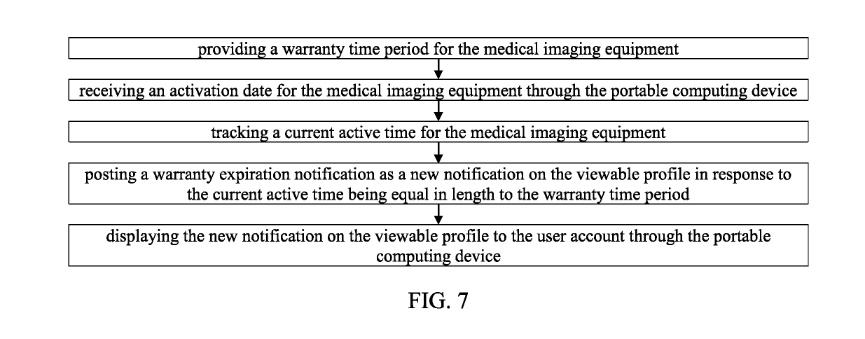

Due to the large number of purchases, the user may have difficulties in remembering important dates for each of the purchases. As illustrated in FIG. 7, the present invention addresses the issue by keeping track of the important dates which can be, but is not limited to, a warranty time period. In order to do so, the warranty time period is provided for the medical imaging equipment. When the user starts utilizing the medical imaging equipment, an activation date for the medical imaging equipment is received through the portable computing device. The activation date notifies the matching manufacturer account about the first day the user starts utilizing the medical imaging equipment. As a result, the matching manufacturer account can use the activation date to determine warranty periods and other dates such as return dates for the medical imaging equipment. In order to do so, the present invention tracks a current active time for the medical imaging equipment. When the current active time is equal in length to the warranty time period, a warranty expiration notification is posted on the viewable profile. However, the present invention can also be configured such that the user account is sent periodic reminders regarding the warranty time period. In order to notify the user account, the new notification is displayed on the viewable profile. Therefore, the user account can view the new notification through the portable computing device.

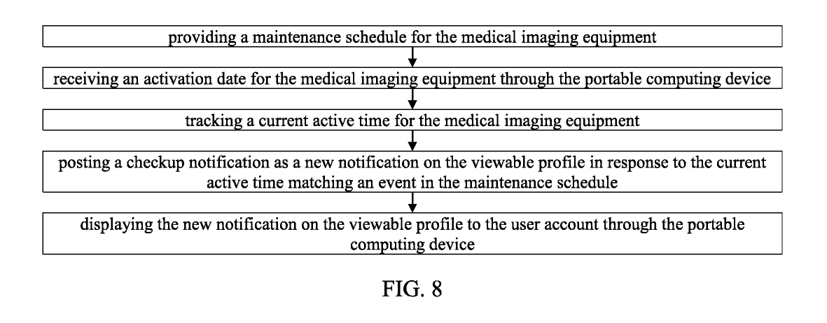

The present invention can also be used to keep track of a list of essential duties specific to the medical imaging equipment as illustrated in FIG. 8. As an example, if we consider the medical imaging equipment to be the magnetic resonance imaging (MRI) equipment, the present invention can be used to keep track and notify the user account regarding the next maintenance date of the magnetic resonance imaging (MRI) equipment. In order to complete such tasks, a maintenance schedule is provided for the medical imaging equipment. The activation date is received through the portable computing device such that the present invention is notified when a particular maintenance task is completed. In order to notify the user account in a timely manner, the present invention tracks the current active time. If the current active time matches an event on the maintenance schedule, a checkup notification is posted as a new notification on the viewable profile. The checkup notification includes all information related to the event on the maintenance schedule. As completed in previous instances, the new notification is displayed on the viewable profile so that the user account can access the new notification through the portable computing device.

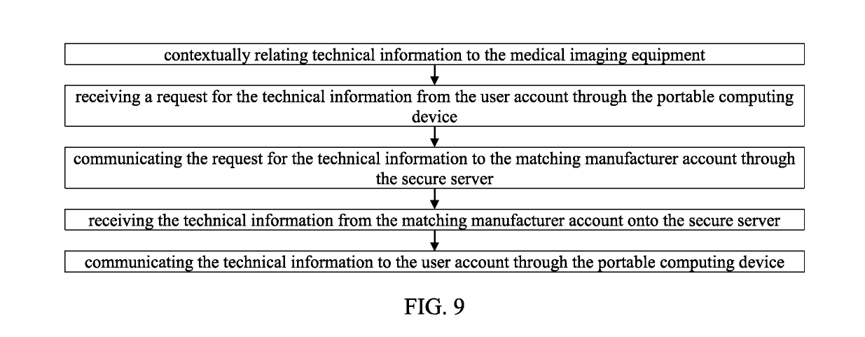

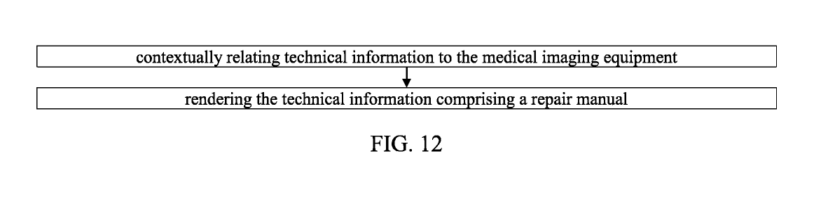

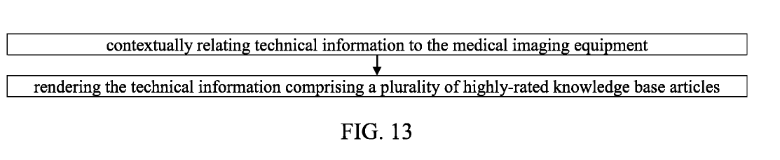

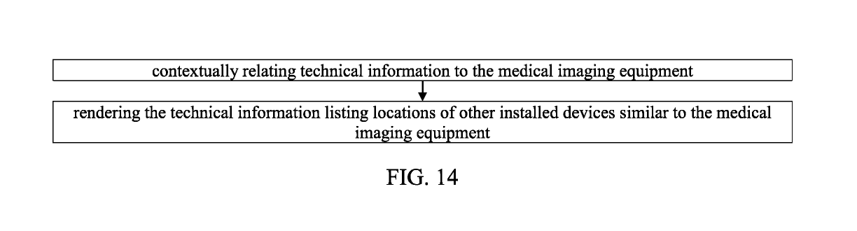

In addition to using the communication line between the user account and the matching manufacturer account for the above-mentioned tasks, the present invention can also be used for providing technical information regarding the medical imaging equipment. As shown in FIG. 9, the present invention initially receives a request for technical information from the user account through the portable computing device. When received, the request for the technical information is communicated to the matching manufacturer account through the secure server such that the user account is guaranteed to get technical information from a reliable source. The present invention receives the technical information from the matching manufacturer account onto the secure server such that the technical information can be promptly forwarded to the user account. More specifically, the technical information is contextually related to the medical imaging equipment such that the request for the technical information is appropriately addressed. In doing so, the present invention communicates the technical information to the user account through the portable computing device. The request for the technical information can vary according to the user's needs. As an example, if the user intends on repairing the medical imaging equipment, the technical information can be a repair manual, as shown in FIG. 12. In another instance, the technical information can list locations of other installed devices similar to the medical imaging equipment, as shown in FIG. 14. Therefore, the user account can utilize the list of locations to examine other installed devices and address the issue of the physical consumer device accordingly. In another instance, the technical information can be a plurality of highly-rated knowledge base articles, as shown in FIG. 13. The plurality of highly-rated knowledge base articles can be used to get additional information regarding the medical imaging equipment. As an example, the user account can utilize the highly-rated knowledge base articles to compare the medical imaging equipment with other comparable products to get a better understanding of the medical imaging equipment.

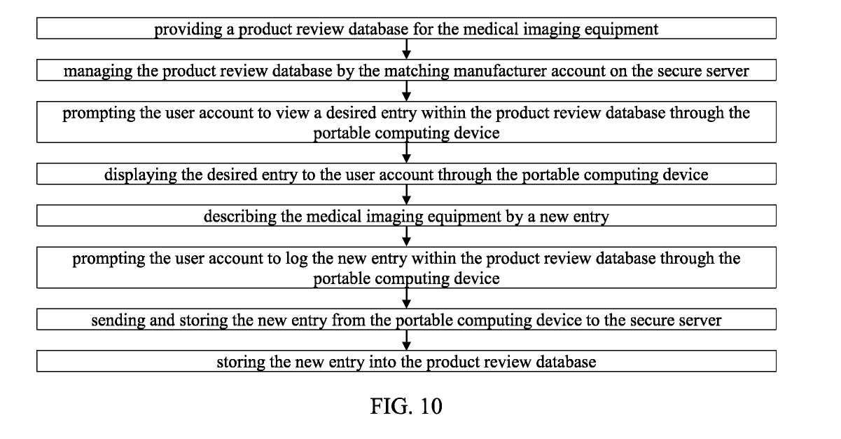

As seen in FIG. 10, the present invention can also be used to read reviews from other users of the medical imaging equipment. In order to do so, a product review database for the medical imaging equipment is provided. The product review database is managed by the matching manufacturer account on the secure server. The user account can both view and also post reviews regarding the medical imaging equipment. In the process of displaying the product review, the user account is initially prompted to view a desired entry within the product review database through the portable computing device. Next, the present invention displays the desired entry within the product review database through the portable computing device. Therefore, the user can get additional information regarding the medical imaging equipment. When the user intends on posting a review, the present invention prompts the user account to log a new entry within the product review database through the portable computing device. More specifically, the new entry describes the medical imaging equipment. When the new entry is received, the present invention sends and stores the new entry from the portable computing device to the secure server. Next, the new entry is stored into the product review database such that other users can view the new entry posted by the user account.

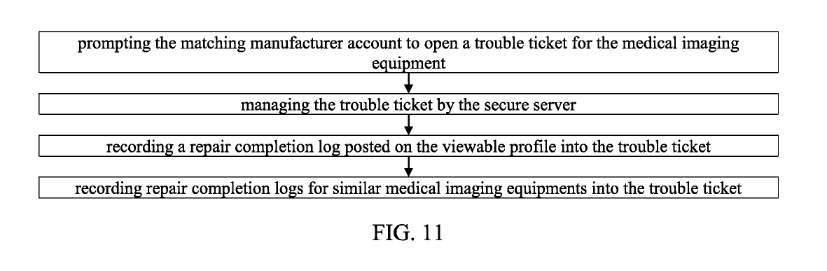

The present invention helps each of the plurality of manufacturer accounts to track the detection, reporting, and resolution of some type of problem. As illustrated in FIG. 11, the present invention prompts the matching manufacturer account to open a trouble ticket, managed with the secure server, for the medical imaging equipment. When the trouble ticket is established, a repair completion log which is posted on the viewable profile is recorded into the trouble ticket. Similarly, repair completion logs for similar medical imaging equipments are recorded into the trouble ticket such that the matching manufacturer account has a clear understanding of the faults of the medical imaging equipment. Therefore, the trouble ticket can be utilized to address an issue of the medical imaging equipment in an effective manner.

In utilizing the present invention, the following process flow can be followed. Firstly, when the user purchases the medical imaging equipment, the user scans the QR code with the portable computing device. When scanned, the identification data of the medical imaging equipment is saved on the secure server. Next, the identification data is compared to each of the plurality of manufacturer accounts in order to find the matching manufacturer account. When the matching manufacturer account is found, the present invention links the user account and the matching manufacturer account. As a result, the matching manufacturer account can push notifications to the user account through the secure server. Recall information, warranty period information, information regarding updates and surveys, technical information, and other comparable information can be sent from the matching manufacturer account to the user account. The manufacturer can also utilize the present invention to promote product development and marketing. Similarly, the user account can contact the matching manufacturer account through the secure server. By contacting the matching manufacturer account, the user account can get support and services related to the medical imaging equipment. Therefore, the present invention can be used to centrally manage all personal items belonging to the user account. Additionally, the user account can also view reviews and other related information of the medical imaging equipment by accessing the secure server. Therefore, the user is guaranteed to make maximum use of the medical imaging equipment by utilizing the present invention to connect to the matching manufacturer account.

Accordingly, the method for providing product information and support services of the present invention creates unique QR code for the medical imaging equipment made by a manufacturing company. The QR code is used to send product support information to a manufacturer and for the manufacturer to check product authenticity.

The method for providing product information and support services of the present invention has several benefits. For example, it helps the manufacturer generate and centrally manage QR codes. QR codes help customers to register their products for customer care and to verify a products authenticity. As products flow across supply chain, distributers and retailers will use QR codes to register their activities. The QR code will route the people to a web portal, or people can view content from an app.

Additionally, depending on the role of the user (manufacturer, distributors, retailers or consumers), different information will be displayed. For example, the purpose of the information will be to provide each level visibility to product issues and customers complaint. Manufacturers can monitor distributers, distributors can monitor retailers, and retailers can monitor customers. Customers can validate a product authenticity because the entire supply chain can be traced back to the manufacturer.

The present invention is important to validate consumers goods, food, medicine. Cad parts are all authentic from manufacturer/producers along with also providing companies information on how products are being used.

Although the invention has been explained in relation to its preferred embodiment, it is to be understood that many other possible modifications and variations can be made without departing from the spirit and scope of the invention as hereinafter claimed.

What is claimed is:

1. A method for providing product information and support services comprising:
providing a medical imaging equipment;
providing a plurality of manufacturer accounts;
providing a user account;
providing a secure server;
managing the plurality of manufacturer accounts and the user account by the secure server;
providing a portable computing device;
communicably linking the user account to the secure server through the portable computing device;
rendering the medical imaging equipment comprising a quick response (QR) code and identification data;
embedding the identification data within the QR code;
rendering the portable computing device comprising a QR code scanner;
receiving the identification data by scanning the QR code with the QR code scanner;
comparing the identification data to each of the plurality of manufacturer accounts in order to identify a matching manufacturer account associated with the medical imaging equipment, the matching manufacturer account being from the plurality of manufacturer accounts;
in response to the matching manufacturer account being identified, communicably linking the user account and the matching manufacturer account through the secure server;
in response to the user account and the matching manufacturer account being communicably linked, performing an authenticity validation for the medical imaging equipment based on the identification data so as to selectively issue a terminating instruction or a proceeding instruction;
in response to the terminating instruction being issued, communicably unlinking the user account and the matching manufacturer account;
in response to the proceeding instruction being issued, generating a viewable profile for the medical imaging equipment on the secure server, and enabling the user account or the matching manufacturer account to update the viewable profile with a notification or a log.

2. The method as claimed in claim 1 comprising:
prompting to photograph the medical imaging equipment through the portable computing device;
rendering the portable computing device comprising a camera;
receiving a visual image of the medical imaging equipment from the camera;
rendering the portable computing device comprising a global positioning system (GPS) locator;
receiving geospatial coordinates for the medical imaging equipment from the GPS locator;
compiling the visual image and the geospatial coordinates into location information; and
displaying the location information as a part of the viewable profile.

3. The method as claimed in claim 1 comprising:
receiving a repair completion log for the medical imaging equipment from the matching manufacturer account;
posting the repair completion log as a new notification on the viewable profile; and
displaying the new notification on the viewable profile to the user account through the portable computing device.

4. The method as claimed in claim 1 comprising:
receiving recall information for the medical imaging equipment from the matching manufacturer account;
posting the recall information as a new notification on the viewable profile; and
displaying the new notification on the viewable profile to the user account through the portable computing device.

5. The method as claimed in claim 1 comprising:
rendering the physical consumer device requiring an operational software;
receiving an update notification for the operational software from the matching manufacturer account;
posting the update notification as a new notification on the viewable profile; and
displaying the new notification on the viewable profile to the user account through the portable computing device.

6. The method as claimed in claim 5 comprising:
prompting to install software upgrades for the operational software through the new notification; and
downloading the software upgrades from the secure server to the portable computing device in response to the user account confirming to install the software upgrades onto the portable computing device.

7. The method as claimed in claim 1 comprising:
providing a warranty time period for the medical imaging equipment;
receiving an activation date for the medical imaging equipment through the portable computing device;
tracking a current active time for the medical imaging equipment;
posting a warranty expiration notification as a new notification on the viewable profile in response to the current active time being equal in length to the warranty time period; and
displaying the new notification on the viewable profile to the user account through the portable computing device.

8. The method as claimed in claim 1 comprising:
providing a maintenance schedule for the medical imaging equipment;
receiving an activation date for the medical imaging equipment through the portable computing device;
tracking a current active time for the medical imaging equipment;
posting a checkup notification as a new notification on the viewable profile in response to the current active time matching an event in the maintenance schedule; and
displaying the new notification on the viewable profile to the user account through the portable computing device.

9. The method as claimed in claim 1 comprising:
contextually relating technical information to the medical imaging equipment;
receiving a request for the technical information from the user account through the portable computing device;
communicating the request for the technical information to the matching manufacturer account through the secure server;
receiving the technical information from the matching manufacturer account onto the secure server; and
communicating the technical information to the user account through the portable computing device.

10. The method as claimed in claim 9 comprising:
rendering the technical information comprising a repair manual.

11. The method as claimed in claim 9 comprising:
rendering the technical information comprising a plurality of highly-rated knowledge base articles.

12. The method as claimed in claim 9 comprising:
rendering the technical information listing locations of other installed devices similar to the medical imaging equipment.

13. The method as claimed in claim 1 comprising:
providing a product review database for the medical imaging equipment;
managing the product review database by the matching manufacturer account on the secure server;
prompting the user account to view a desired entry within the product review database through the portable computing device;
displaying the desired entry to the user account through the portable computing device;
describing the medical imaging equipment by a new entry;
prompting the user account to log the new entry within the product review database through the portable computing device;
sending and storing the new entry from the portable computing device to the secure server; and
storing the new entry into the product review database.

14. The method as claimed in claim 1 comprising:
prompting the matching manufacturer account to open a trouble ticket for the medical imaging equipment;
managing the trouble ticket by the secure server;
recording a repair completion log posted on the viewable profile into the trouble ticket; and
recording repair completion logs for similar medical imaging equipments into the trouble ticket.

15. The method as claimed in claim 1 comprising:
providing a magnetic resonance imaging (MRI) equipment as the medical imaging equipment.
